(19) United States
(12) Patent Application Publication
(10) Pub. No.: US 2011/0293518 A1
(43) Pub. Date: Dec. 1, 2011

(54) DIAGNOSIS OF WHIPLASH ASSOCIATED DISORDERS (WAD) BY USING PET WITH D-[METHYL-11C]-DEPRENYL (DDE)

(75) Inventors: Henry Engler, Uppsala (SE); Bengt Langstrom, Uppsala (SE); Mats Bergstrom, Basel (CH); Lieuwe Appel, Uppsala (SE); Mats Fredrikson, Uppsala (SE)

(73) Assignee: GE HEALTHCARE LIMITED, Little Chalfont (GB)

(21) Appl. No.: 13/183,530

(22) Filed: Jul. 15, 2011

Related U.S. Application Data
(63) Continuation of application No. 12/065,103, filed on Aug. 27, 2008.
(60) Provisional application No. 60/712,875, filed on Aug. 31, 2005.

(51) Int. Cl. A61K 51/04

(52) U.S. Cl. 424/1.81

(57) ABSTRACT

Positron Emission Tomography (PET) tracers such as D-[methyl-¹¹C]-Deprenyl (DDE) and [¹¹C]-GR205171 (GLD), methods for and methods of preparing biological mechanisms that identify treatment targets in connection with Whiplash-Associated Disorder (WAD) are provided. Associated kits for the evaluation of the biological mechanisms are also provided.

### FIELD OF THE INVENTION

The present invention relates to the development of a Positron Emission Turnover (PET)-tracer that could be used to diagnose a whiplash-associated disorder (WAD). The present invention further relates to methods for the diagnostic use of a PET tracer that can be used for WAD, The present invention further relates to the studies of underlying biological mechanisms that could contribute to identifying new diagnosis and treatment targets for WADs.

### BACKGROUND OF THE INVENTION

The commonly used term Whiplash is defined as an acceleration-deceleration mechanism of energy transfer to the neck which may result from rear-end or side impact, predominately in motor vehicle accidents and from other mishaps. The energy transfer may result in bony or soft tissue injuries (whiplash injury) which may in turn lead to a wide variety of clinical manifestations such as whiplash associated disorders. (Spitzer W O, Skovron M L, Salmi L R et al., Scientific monograph of the Quebec Task Force on WAD: redefining whiplash and its management”, Spine, 1995).

Whiplash injuries are common; the incidence has been estimated to be approximately 4 per 1000 people (Barnsley L, Lord S., Bogduk N., “Whiplash injury”, Pain, 1994). Although many people involved in motor vehicle accidents recover quickly, between 4% and 42% of patients with accident-related neck injuries report symptoms several years later. (Lord S M, Barnsley L., Wallis B J, Bogduk N., “Chronic cervical zygapophysial joint pain after whiplash: a placebo-controlled prevalence study”, Spine, 1996).

Chronic pain syndromes which include whiplash-associated disorders (WAD) are prevalent, cause significant individual suffering, and are high societal costs. In contrast to other injuries due to traffic accidents, neck injuries have increased. For example, neck injuries associated with at least 10% disability have increased from roughly 30 to roughly 60% during the two latest decades.

Like chronic pain syndromes, WAD causes significant individual suffering as well as high societal costs. For example, over 29 billion dollars per year is spent on whiplash injuries and litigation in the United States. Roughly ⅓rd of all WAD patients develop chronic problems. (Lord SM, Barnsley L., Wallis B J, Bogduk N., “Chronic cervical zygapophysial joint pain after whiplash: a placebo-controlled prevalence study”, Spine, 1996).

A continuous search is under way for new treatment modalities of WAD, but at present, much work is focused on the early identification of patients with WAD. Although generally accepted diagnostic criteria for WAD as well as outcome measures exist, prognostic markers are lacking. Present methods do not allow important subgroups of patients to be clearly distinguished. The need for more precise methods to determine severity and prognosis as well as a treatment response is urgent. Conventional radiology only gives information about already established injuries. Magnetic Resonance Imaging tomography (MRI), computer tomography (CT) and radioisotope methods have shown potentials of visualizing signs of biological processes, but more work is needed before they can be used as an objective and quantitative assessment of WAD and different therapeutic strategies.

Positron emission tomography (PET) imaging is not currently used in the diagnosis of WAD, although PET has been used to demonstrate high glucose metabolism in joints of rheumatoid arthritis patients. PET has also been used in conjunction with the tracer D-[methyl-¹¹C]-deprenyl (also known as DDE or ¹¹C-D-deprenyl) in diagnosing and treating patients with arthritis. (Danfors T., Bergstrom, M. et al., “Positron Emission Tomography with ¹¹C-D-deprenyl in Patients with Rheumatoid Arthritis”, Scan J. Rheumatol, 1997).

Furthermore, DDE has been used as a negative control for the demonstration of selective MAO-B-binding by ¹¹C-L-deprenyl. ¹¹C-L-deprenyl is an enantiomer of DDE. An enantiomer exists when a chemical structure and its mirror image are not superposable. In a range of pituitary adenomas, ¹¹C-L-deprenyl showed significant retention in both a tumourous and normal brain, whereas in most of the tumours, DDE showed a rapid washout from both tumour and normal brain. However, in some tumours, DDE was retained in the tumour but not in the normal brain. The washout suggested that DDE had negligible binding to MAO-B, but the retention in some tumours suggested an additional mechanism of retention ofDDE. (Danfors T., Bergstrom, M. et al., “Positron Emission Tomography with ¹¹C-D-deprenyl in Patients with Rheumatoid Arthritis”, Scan J. Rheumatol, 1997). MAO-B is monoamine oxidase B which is identified as a member of the family of the imidazoline binding proteins.

Moreover, there has yet to be found a PET-tracer that could be used for WAD. Accordingly, there has been a long felt need for the development of a PET-tracer that could be used to diagnose WAD as well as study underlying biological mechanisms that retain a PET-tracer which could contribute to identifying new diagnosis and treatment targets for WAD.

Discussion or citation of a reference herein shall not be construed as an admission that such reference is prior art to the present invention.

### SUMMARY OF THE INVENTION

In view of the long felt need for diagnosing whiplash-associated disorder (WAD), the present invention relates to both the development of a PET-tracer that could be used to diagnose WAD and the study of underlying biological mechanisms that could contribute to identifying new diagnosis and treatment targets for WADs.

In one embodiment, a Positron Emission Tomograhpy (PET) tracer for diagnosing whiplash-associated disorder is disclosed wherein the PET tracer is D-[methyl-¹¹C]-Deprenyl DDE). The PET tracer could also be [¹¹C]-GR205171 (GLD).

In a further embodiment, a biological mechanism that is a specific molecular structure or enzyme which is expressed in the inflammed joint tissue, wherein the biological mechanism could contribute to identifying treatment targets according to WADs is also disclosed.

In another embodiment, a method for the preparation of an enzyme or molecular structure according to the steps of using capillary electrophoresis to aid in separating out proteins such as MAO-B proteins; and then detecting the radioacitivity by ¹¹C-labelled precursors to guide a skilled artisan as to which MAO-B protein would bind to either DDE and GLD is also presented.

The present invention further provides for a kit in the preparation of a molecular structure or enzyme according to the steps of using capillary electrophoresis to aid in separating out proteins such as MAO-B proteins; and then detecting the radioacitivity by ¹¹C-labelled precursors to guide which MAO-B protein would bind to either DDE and GLD.

### BRIEF DESCRIPTION OF THE FIGURES

FIG. 1 shows a PET-imaging picture that depicts an increased DDE uptake in one of the WAD patients.

### DETAILED DESCRIPTION OF THE INVENTION

The present invention relates to examining patients with Whiplash associated-syndrome (WAD) by investigating the presence of local changes, caused by local micro-lesions and producing an increased ¹¹C-D-deprenyl (DDE) uptake revealed through Positron Emission Tomography (PET) that is impossible to be revealed by computer tomography (CT) or Magnetic Resonance Imaging tomography (MRI).

PET imaging is a tomographic nuclear imaging technique that uses radioactive tracer molecules that emit positrons. When a positron meets an electron, they both are annihilated and the result is a release of energy in the form of gamma rays, which are detected by the PET scanner. By employing natural substances that are used by the body as tracer molecules, PET does not only provide information about structures in the body but also information about the physiological function of the body or certain areas therein. Furthermore, the choice of a tracer molecule depends on what is being scanned. Generally, a tracer is chosen that will accumulate in the area of interest, or be selectively taken up by a certain type of tissue, e.g. cancer cells. Scanning consists of either a dynamic series or a static image obtained after an interval during which the radioactive tracer molecule enters the biochemical process of interest. The scanner detects the spatial and temporal distribution of the tracer molecule. PET also is a quantitative imaging method allowing the measurement of regional concentrations of the radioactive tracer molecule. Commonly used radionuclides in PET tracers are ¹¹C, ¹⁸F, ¹⁵O ¹³N or ⁷⁶Br.

Furthermore, tracers labeled with short-lived positron emitting radionuclides (e.g. ¹¹C, t_{1/2}=20.3 min) are frequently used in various non-invasive in vivo studies in combination with PET. Because of the radioactivity, the short half-lives and the submicromolar amounts of the labeled substances, extraordinary synthetic procedures are required for the production of these tracers. An important part of the elaboration of these procedures is the development and handling of new ¹¹C-labelled precursors. This is important not only for labeling new types of compounds, but also for increasing the possibility of labeling a given compound in different positions.

When compounds are labeled with ¹¹C, it is usually important to maximize specific radioactivity. In order to achieve this, the isotopic dilution and the synthesis time must be minimized. Isotopic dilution from atmospheric carbon dioxide may be substantial when [¹¹C]carbon dioxide is used in a labeling reaction. Due to the low reactivity and atmospheric concentration of carbon monoxide (0.1 ppm vs. 3.4×10⁴ ppm for CO₂), this problem is reduced with reactions using [¹¹C]carbon monoxide.

There are several advantages in using the PET technique in the diagnosis of WAD. One advantage is that the PET technique offers a potential for recording various functional and biochemical characteristics in the affected joints. Another advantage is that the PET technique has a potential to supply objective and quantitative estimates of disease intensity rather than secondary structural information.

One embodiment of the invention is to provide a Positron Emission Tomograhpy (PET) tracer for diagnosing whiplash-associated disorder wherein a PET tracer is D-[methyl-11-C]-Deprenyl (DDE). The PET tracer can also be [11C]-GR205171 (GLD).

Another embodiment of the present invention includes either DDE or GLD or in combination thereof can be used for joint diseases comprising arthritis, rheumatoid arthritis, gout, osteoarthritis, ankylosis, bursitis, temporomandibular joint disorders, synovial chrondromatosis, hemarthrosis, acquired joint deformalities, metatarsalgia, arthralgia, arthrogryposis, joint instability, synovitis, neurogenic arthropathy, hallux rigidus, hydrathrosis, joint loose bodies, and similar diseases thereof.

In a further embodiment, a biological mechanism that is a specific molecular structure or enzyme which is expressed in the inflammed joint tissue of a patient, wherein the biological mechanism identifies treatment targets according to WADs is also disclosed. A patient used herein is a human being or any kind of animal thereof.

In yet another embodiment, a method for the preparation of a molecular structure or an enzyme according to the steps:
i) using capillary electrophoresis to aid in separating out MAO-B proteins; and then
ii) detecting the radioactivity by ¹¹C-labelled precursors to guide a skilled artisan as to which MAO-B protein would bind to either or both DDE and GLD is also presented. A skilled artisan used throughout this application is defined as a person who is of skill in this particular field.

The present invention further provides a kit for the preparation of a molecular structure or an enzyme according to the steps:
i) using capillary electrophoresis to aid in separating out MAO-B proteins; and then
ii) detecting the radioactivity by ¹¹C-labelled precursors to guide a skilled artisan as to which MAO-B protein would bind to either or both DDE and GLD is also presented.

Yet another embodiment of the present invention provides for a diagnostic use of a PET tracer comprising D-[methyl-11-C]-Deprenyl (“DDE”) or [11C]-GR205171 (“GLD”) for determining WAD.

Still a further embodiment encompasses a method of use for generating a biological mechanism that is a specific molecular structure which is expressed in the inflammed joint tissue of a patient, wherein the biological mechanism identifies treatment targets according to WADs.

While still another embodiment entails a method of use for generating a biological mechanism that is an enzyme which is expressed in the inflammed joint tissue of a patient, wherein the biological mechanism identifies treatment targets according to WADs.

Another embodiment of the present invention entails a method of use for preparing a molecular structure according to the steps:
i) using capillary electrophoresis to aid in separating out MAO-B proteins; and then ii) detecting the radioactivity by ¹¹C-labelled precursors, whereby guiding the MAO-B proteins to either bind to DDE, GLD or both.

A further embodiment of the present invention encompasses a method of use for preparing an enzyme according to the steps:
i) using capillary electrophoresis to aid in separating out MAO-B proteins; and then
ii) detecting the radioactivity by ¹¹C-labelled precursors, whereby guiding the MAO-B proteins to either bind to DDE, GLD or both.

### EXAMPLES

The invention is further described in the following examples which are in no way intended to limit the scope of the invention.

Uppsala Imanet has developed two tracers which are D-[methyl-¹¹C]-deprenyl (DDE) and [11C]-GR205171 (GLD) that are to be used in combination with a Positron Emmison Tomography (PET) scanner, for clinical investigations indicating an inflammatory process such as Whiplash Associated-Disorder (WAD).

In one experiment, eight patients have been examined and diagnosed with WAD through the use of DDE and PET. In four patients DDE appeared to bind to soft tissue in the neck vertebra. Clinically, those patients with a higher uptake of DDE in the affected joint areas seem to report high pain ratings while being in the scanner.

Furthermore, when a patient was administered intra-articular injections of gluccorticoids a significant diminishing of the uptake of DDE in the affected area, such as but not limited to the synovial (or diarthrodial) joint or similar joints, was observed 6-14 days after treatment. The effects are observed with all modes of evaluation: After treatment, the intitial high uptake of DDE in the affected joint is significantly diminished, indicating a pronounced effect on perfusion.

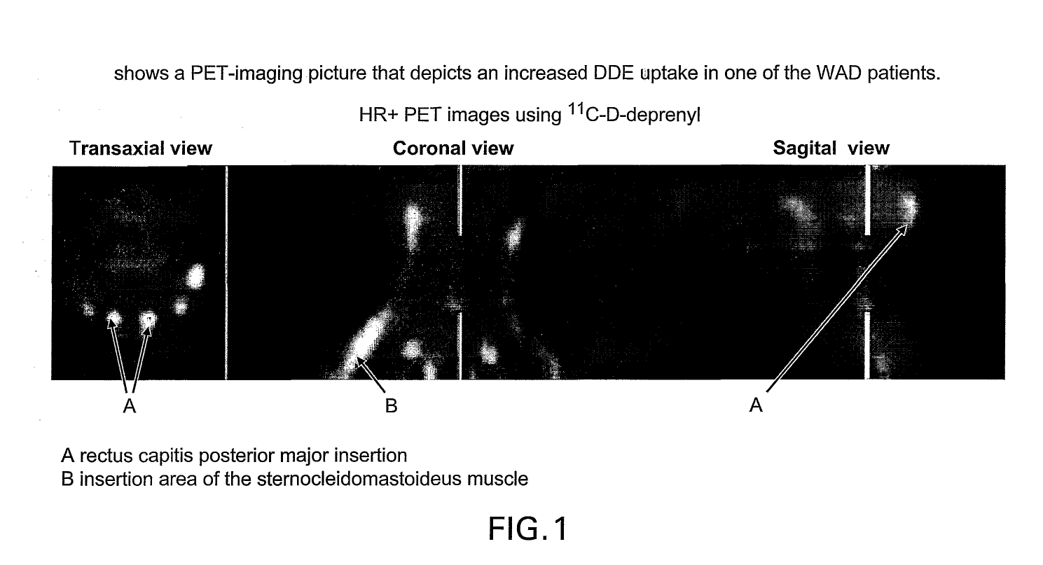

In a similar experiment, sixteen patients were investigated using a high resolution PET scanner. It was observed that about 50% of the patients had an increased uptake of DDE in the neck and shoulder areas of the patients. As an example, FIG. 1 shows a PET-imaging picture that depicts an increased DDE uptake in one of the WAD patients.

In a third experiment eight healthy subjects were investigated using a PET-CT (Computerized Tomography) scanner. PET/CT combines the strengths of two well-established imaging modalities, PET for function and CT for anatomy, into a single imaging device. The subjects were free from pain in the neck and shoulder regions during at least two months and they had not been involved in accidents which could have caused a neck injury. None of the subjects revealed an increased uptake of DDE in the scanned neck and shoulder regions. This indicates that the increased uptake in patients (experiments 1 and 2) are related to their clinical symptoms.

Furthermore, on-going analysis are being performed through semi-quantitative analysis by comparing the neck and shoulder areas wih an increased DDE uptake to an area of the body without any DDE uptake. Additionally, a control material is planned to compare WAD-patients and healthy volunteers.

In search for a mechanism of retaining DDE, studies regarding inflamed joints such as synovial (or diarthrodial) were adminstered. The results uncovered a very pronounced uptake and retention on swollen synovial tissue, correlating well with clinical and others signs of active inflammation. In a separate study, an investigation pertaining to the radioactivity concentration in synovial fluid of patients with rheumatoid arthritis given the tracer DDE was adminstered. The synovial fluid had low radioactivity whereas the inflamed tissue as seen in the PET images had very high activity. This activity was reduced by 50% the day after intra-articular administration of glucocorticoid.

There are various hypothesis as to what biological mechanisms are behind the high uptake of DDE in inflammatory joint tissues of humans. One hypothesis is binding DDE to a specific molecular structure or enzyme which is expressed in the inflamed area. To validate the hypothesis a frozen section autoradiography was performed on various tissues using DDE and thereby demonstrating a much higher binding in inflamed joints than any other tissues. In further determining the character of the binding between DDE and inflammatory joint tissues, it is anticipated that capillary electrophoresis would aid in separating out proteins and the detection of the radioactivity would guide one to know how many and potentially which protein is binding to the tracer.

In addition, in order to exclude the possibility that the DDE uptake is governed by binding to a monoamine oxidase B (MAO-B), a reexamination of one patient after adminstration of Eldepryl demonstrated to be sufficient for blocking of an enzyme. In this patient no blocking effect could be seen, only a slightly higher uptake of DDE in the inflamed joint area. In the human brain and pituitary adenomas it has been shown that ¹¹C-L-deprenyl has a higher binding, whereas DDE is rapidly washed out except in subgroups of non-secreting pituitary adenomas where DDE shows high binding.

Furthermore, it has been hypothesized that the pharmacological challenge of the binding between DDE and inflammatory joint tissues in patients is when inflamed synovial tissue is incubated with DDE, with or without a range of MAO-B binding proteins or similar compounds which could potentially inhibit the binding. The inhibitors that could be used to potentially inhibit the binding between DDE and inflammatory joint tissue are inhibitors of VAP-1 and other cell surface amino oxidases. Additionally, other molecular entities are searched for which would serve the same task of binding to the identified molecular target.

In addtion, in a small pilot series of experiments in patients with bacterial abscesses, no visible increase of DDE or GLD uptake was found.

Furthermore, in vitro binding experiments demonstrated very high DDE and GLD tracer binding in inflammatory tissues removed from patients with rheumatoid arthritis. The in vitro binding experiments were performed on thin slices of tissue of about 20 micrometers in rats and in humans. The thin slices of tissues were adhered to gelatinized slide glasses. The tissue adhered to the glass was then incubated in a buffer such as TRIS-HCl containing DDE or GLD at about 2 nM concentration for about 40 minutes at room temperature. The experimental conditions were varied and the results were not significantly affected. After incubation the slide glasses were washed about 3 times in a buffer, dried, and exposed on phosphor imaging plates for a minimum of 60 minutes.

In the in vitro binding experiments, local uptake was present in all cases in the parotis and submandibularis glandulae. Differences between patients were found in areas related to the insertion of muscles to the occipital bone, vertebrae and clavicula. In these regions, four of the examined patients showed a visually enhanced uptake in comparison with the rest. In some of these patients an increased uptake was observed in de proximal and distal parts of the muscle sternocleidomastoideus.

In order to make inter-individual comparisons, it was decided to normalize the uptake in the above-mentioned areas with the uptake of the inferior part of the cerebellum. In the area of insertion of m. rectus capitis posterior major we found bilaterally in 3 patients an increase of 57%-127% in the uptake of DDE compared with the cerebellum. The fourth patient did not show a clear increase, but there was a 40% difference between right and left side of the cerebellum.

At the level of the cervical vertebrae 2 and 3 (insertion in m. semispinalis capitis) it was found in one case an increase of approximately 70% uptake of DDE and GLD. The other two patients showed lower uptake of these two PET tracers but differences in the patients were found between both sides of the cervical vertebrae in the order of 16-30%. High uptake of DDE and GLD at the level of insertion in the clavicula 70-120% was found in some of these four patients.

### Specific Embodiments, Citation of References

The present invention is not to be limited in scope by specific embodiments described herein. Indeed, various modifications of the inventions in addition to those described herein will become apparent to those skilled in the art from the foregoing description and accompanying figures. Such modifications are intended to fall within the scope of the claims.

Various publications and patent applications are cited herein, the disclosures of which are incorporated by reference in their entireties.

1. A method for diagnosing whiplash-associated disorder (WAD) in a subject comprising:
(a) administering an effective amount of D-[methyl-¹¹C]-deprenyl (DDE) to a subject; and
(b) performing a PET scan on said subject.

2. (canceled)

3. A PET tracer according to claim 1, wherein the PET tracer comprises [11C]-GR205171 (GLD).

4-15. (canceled)

16. The method of claim 1, wherein said PET scan comprises a PET-CT scan.

17. The method of claim 1, wherein said PET scan is performed on the neck region of said subject.

18. The method of claim 17, wherein said PET scan shows an increase in DDE uptake in the neck region of said subject.

19. The method of claim 1, wherein said PET scan is performed on the shoulder region of said subject.

20. The method of claim 19, wherein said PET scan shows an increase in DDE uptake in the shoulder region of said subject.

21. A method for PET imaging a subject believed to be suffering from whiplash-associated disorder (WAD), the method comprising:
(a) administering an effective amount of D-[methyl-¹¹C]-deprenyl (DDE) to a subject; and
(b) performing a PET scan on said subject.

22. The method of claim 21, wherein said PET scan comprises a PET-CT scan.

23. The method of claim 21, wherein said PET scan is performed on the neck region of said subject.

24. The method of claim 23, wherein said PET scan shows an increase in DDE uptake in the neck region of said subject.

25. The method of claim 21, wherein said PET scan is performed on the shoulder region of said subject.

26. The method of claim 25, wherein said PET scan shows an increase in DDE uptake in the shoulder region of said subject.

27. A method for diagnosing whiplash-associated disorder (WAD) in a subject comprising:
(a) administering an effective amount of [11C]-GR205171 (GLD) to a subject; and
(b) performing a PET scan on said subject.
